(19) United States
(12) Patent Application Publication
(10) Pub. No.: US 2010/0240674 A1
(43) Pub. Date: Sep. 23, 2010

(54) USE OF COMPOUNDS DERIVED FROM BENZAMIDINE METHANESULPHONATE OR A PHARMACEUTICALLY ACCEPTABLE SALT THEREOF, FOR EXAMPLE IMATINIB MESYLATE, FOR THE PREPARATION O F A DRUG FOR THE TREATMENT OF NEURODEGENERATIVE NIEMANN-PICK C DISEASE (NPC)

(75) Inventors: Silvana Zanlungo Matsuhiro, Santiago de Chile (CL); Francisca Bronfman Caceres, Santiago de Chile (CL); Alejandra Alvarez Rojas, Santiago de Chile (CL)

Correspondence Address: MERCHANT & GOULD PC, P.O. BOX 2903, MINNEAPOLIS, MN, 55402-0903, US

(73) Assignee: PONTIFICIA UNIVERSIDAD CATOLICA DE CHILE, Santiago de Chile (CL)

(21) Appl. No.: 12/602,447

(22) PCT Filed: May 28, 2008
(86) PCT No.: PCT/ES2008/000378

§ 371 Date: Jun. 4, 2010

(30) Foreign Application Priority Data
May 28, 2007 (CL) 152-2007

(51) Int. Cl. A61K 31/506; A61P 25/00; A61P 3/00

(52) U.S. Cl. 514/252.18

(57) ABSTRACT

This invention relates to the use of imatinib mesylate for the treatment of metabolism disorders characterized by neurodegeneration, in particular a metabolism disorder characterized by an inability to metabolize lipids such as in persons with Niemann-Pick Type C disease. More specifically, this invention relates to a method of treating neurodegenerative disease, comprising administering a therapeutically effective amount of a compound derived from imatinib mesylate or a pharmaceutically acceptable salt thereof.

### AIM OF THE INVENTION

This invention relates to the use of imatinib mesylate or a pharmaceutically acceptable salt thereof for the treatment or prevention of neurodegenerative disease, in particular for the treatment or prevention of lipidosis in Niemann-Pick Disease Type C.

### BACKGROUND OF THE INVENTION

Niemann-Pick disease type C(NPC), is a fatal recessive autosomal illness characterized by a dramatic lipidosis. Patients with NPC disease show hepatomegaly and progressive neurodegeneration that ends in early death (Pentchev et al., 1995; Sturley et al., 2004). The cellular phenotype characteristic of this disease is the accumulation of free cholesterol derived from low density lipoproteins (LDL) in the endosomal/lysosomal system (Pentchev et al., 1995; Liscum and Klasek, 1998). The fibroblasts of NPC disease patients exhibit a defect in the pathway by which cholesterol exits from the lysosomes of the cells. At the same time, the fibroblasts of NPC disease patients experience an attenuation in the expression of two key proteins in cholesterol homeostasis: the key enzyme involved in the synthesis of cholesterol, HMG-CoA reductase, and the LDL endocytic receptor, LDLR (Liscum and Faust, 1987; Frolov et al., 2003; Sturley et al., 2004). These factors cause a paradox, because while the NPC disease cells (fibroblasts) are full of cholesterol, they are unable to use or sense the presence of cholesterol, and as a result the cells respond as they would in the absence of cholesterol, increasing the synthesis and uptake of cholesterol from lipoproteins.

There are two identified genes that are determinant in the development of NPC disease: NPC1 and NPC2 (Steinberg et al., 1996). Mutations in the NPC1 gene cause about 90 to 95% of the instances of NPC disease (Carstea et al., 1997). Mutations in the NPC2 gene cause the remainder of the cases (Naureckiene et al., 2000).

The consequences of the lack of expression and/or mutations in the NPC1/2 genes in Central Nervous System (CNS) pathology in NPC disease have been well documented (Walkey and Suzuki, 2004; Paul et al., 2004). As in other cells of the organism, the brain cells accumulate massive amounts of cholesterol and glycosphyngolipids (Reid et al., 2004; Walkey and Suzuki, 2004). However, the brain seems to be especially sensitive to this kind of lipidosis, and as time passes, local damage by the formation of axonal spheroids begins to appear while in advanced stages of NPC disease there is clear neurodegeneration (Walkey and Suzuli, 2004; Paul et al., 2004). Neurodegeneration is especially prevalent in certain brain regions such as in the Purkinje cells in the cerebellum and in the neurons in the hippocampus (Pentchev et al., 1995; Paul et al., 2004; Walkey and Suzuki, 2004). In fact, the clinical symptoms of NPC disease such as progressive ataxia, dystonia, and lack of general locomotor coordination correlate well with the loss of cerebellar Purkinje neurons.

The mechanism by which neurons degenerate in NPC disease is not clear. It is not known how the accumulation of lipids is related to neuronal loss in this disease or in other diseases in which lipidosis occurs. Neuronal death as a result of NPC disease appears to occur by a mechanism of programmed cell death known as apoptosis (Walkey and Suzuli, 2004; Paul et al., 2004; Li et al., 2005; Wu et al., 2005; see also FIG. 5). The inhibition of intracellular cholesterol transport would activate this program of cell death (Huang et al., 2006). In fact, it has been found that there is an increase of a series of apoptotic markers in mice with NPC disease (Li et al., 2005).

Currently there is no cure or treatment for people with NPC disease. As a result, there is a need for treatment of NPC disease, including alleviation of symptoms and attenuation of the progression of the disease.

### SUMMARY OF THE INVENTION

This invention relates to the use of imatinib mesylate for the treatment of metabolism disorders characterized by neurodegeneration, in particular a metabolism disorder characterized by an inability to metabolize lipids such as in persons with Niemann-Pick Type C disease. More specifically, this invention relates to a method of treating neurodegenerative disease, comprising administering a therapeutically effective amount of a compound derived from benzamidine methanesulfonate or a pharmaceutically acceptable salt thereof. This invention also relates to a method of treating neurodegenerative disease, comprising administering a therapeutically effective amount of a compound derived from imatinib mesylate or a pharmaceutically acceptable salt thereof.

### BRIEF DESCRIPTION OF THE FIGURES

FIG. 1 is a series of microphotographs of cerebellum cross sections stained for c-Abl and p73 detection using immunoperoxidase staining from wild type mice and from mice with NPC disease at different ages (4, 6, 8 weeks) showing the expression of the proteins p73 and c-Abl, including an immunohistochemical analysis of p73 (a-f) and c-Abl (g-l) in cerebellums of wild type mice in a, c, e, g, i and k, and in NPC mice in b, d, f, h, j and k.

FIGS. 2A and 2B are graphs showing the effect of treatment with imatinib mesylate on the weight of mice with NPC disease.

FIG. 3 is a graph showing the effect of imatinib mesylate treatment of mice with NPC disease on the score of the locomotor test known as the “Hanging Test.”

FIG. 4 is a series of microphotgraphs of cerebellum cross sections stained to detect calbindin using immunoperoxidase staining from wild type mice and from mice with NPC disease that illustrate the effect of imatinib mesylate treatment on the survival of cerebellar Purkinje neurons in mice with NPC disease.

FIG. 5 is a series of microphotographs of cerebellum cross sections measured using the TUNEL technique that illustrate the effect of imatinib mesylate on the levels of apoptotic death in mice with NPC disease and their correlation with the number of Purkinje neurons.

### DETAILED DESCRIPTION OF THE INVENTION

This invention discloses a new application for a compound derived from benzamidine methanesulfonate or its acceptable pharmaceutical salt to treat or prevent neurodegenerative diseases. A derivative of benzamidine methanesulfonate, imatinib mesylate, was designed to inhibit the proliferation of cancerous cells, because its target is an aberrant c-Abl protein generated in this disease. In this invention, however, it is shown that the use of derivatives of benzamidine methanesulfonate also inhibits the normal c-Abl form and can reduce neuronal death.

The methods used to obtain derivatives of pirimidine or of imatinib mesylate are known in the art. For example, U.S. Pat. No. 5,521,184, European Patent No. 0564409, and Chilean Patent No. 41,937 disclose a crystalline form of imatinib mesylate, 4-[(4-methylpiperazin-1-ylmethyl)-N-[4-methyl-3-(4-pyridin-3-yl)(pyrimidin-2-ylamino)phenyl]benzamide methanesulfonate) U.S. Pat. No. 6,894,051 also discloses a new crystalline form of the methanesulfonic acid addition salt of 4-(4-methylpiperazin-1-ylmethyl)-N-[4-methyl-3-(4-pyridin-3-yl)(pyrimidin-2-ylamino) phenyl]benzamide.

In addition, Chilean patent application number CL1355-2005, published on Dec. 23, 2005 discloses the use of imatinib for the treatment of viral liver diseases and in particular, viral hepatitis. This Chilean patent application also discloses the use of imatinib for the inhibition of the replication and/or transmission of other viruses including herpes, poxvirus, influenza, parainfluenza, respiratory syncytial, rhinovirus, yellow fever, West Nile, and viral encephalitis.

Imatinib mesylate was developed by Novartis and is commercially known as Gleevec®. Imatinib mesylate is indicated for the treatment of chronic myeloid leukemia (CML) and gastrointestinal stromal tumors (GIST). Imatinib mesylate's molecular target is BCR-cAbl, the oncogenic form of the tyrosine kinase c-Abl, and can inhibit the normal as well as the oncogenic c-Abl form.

As described below, studies show that the normal form of c-Abl participates in cellular processes leading to neuronal death in neurodegenerative diseases such as NPC disease or Alzheimer's disease. Derivatives of benzamidine methanesulfonate can therefore be used to attenuate the progression of neurodegenerative diseases. In particular, it has been found that imatinib mesylate is useful in treating the neuropathology in NPC disease. The neuroprotective effect of imatinib mesylate could be extended to other neurodegenerative diseases, such as Alzheimer's disease and Parkinson's disease, due to the similarities seen in these diseases to the cellular changes exhibited in NPC disease.

The following examples are included so as to provide those of ordinary skill in the art with a complete disclosure and description and are not intended to limit the scope of the invention nor are the examples intended to represent that these were the only experiments performed.

The experiments included herein were performed with imatinib mesylate, a drug sold by Novartis® with the chemical name 4-(4-methylpiperazin-1-ylmethyl)-N-[4-methyl-3-((4-pyridin-3-yl)(pyrimidin-2-ylamino)phenyl]benzamide methanesulfonate). Imatinib mesylate is a white-yellow crystalline powder. Its molecular formula is C₂₉H₃₁N₇O.CH₄SO₃. It has a molecular weight of 589.7 g/mol, and it is soluble in aqueous solutions with a pH of about 5.5. Imatinib mesylate is an inhibitor of the Bcr-c-Abl protein kinase, a constitutively active kinase due to the chromosomal translocation 9:22. The Bcr-c-Abl protein kinase forms the Philadelphia chromosome associated with chronic myeloid leukemia (CML). Imatinib mesylate inhibits proliferation and induces apoptosis in cell lines positive for Bcr-c-Abl, thereby providing a treatment for CML. In vivo, imatinib mesylate inhibits tumor growth in CML patients in blastic crisis. Imatinib mesylate also inhibits proliferation and induces apoptosis of gastrointestinal stromal tumor cells (GIST) that express a mutated form of the constitutively activated kinase c-kit.

For both CML and GIST, in which imatinib mesylate is used as a therapeutic agent, the inhibitor target proteins are constitutively activated mutant proteins with activity associated with abnormal cell proliferation. In the invention described herein, however, the target of the inhibitor imatinib mesylate is the normal c-Abl protein kinase (non-mutated) in neurons, differentiated cells of the nervous system. The protein kinase c-Abl is a highly regulated kinase that participates in apoptosis. Even though the c-Abl kinase has been previously described as a target of benzamidine methanesulfonate derivatives, the use of this drug to attempt stopping neuronal death in neurodegenerative disease has not been proposed. Furthermore, previous to the Alvarez et al., 2004 study, the ability of imatinib mesylate to prevent neuronal death had not been described. Moreover, c-Abl function in neurons had been discarded a priori.

The family of transcription factors p53 has been implicated in the apoptosis that takes place in the brains of mice with NPC disease. Specifically, p73, a family member of p53, has been implicated in this apoptosis. For example, there is increasing evidence that the p53 family of transcription factors are key to neuronal survival (Jacobs et al., 2004). Moreover, p73 has been implicated in cell death induced by neuronal damage (Jacobs et al., 2004). Interestingly, through the use of alternative promoters of the p73 gene, two isoforms of p73 with opposite functions in the central nervous system (CNS) can be generated: Delta N-p73 has a pro-survival role while the complete form TAp73 is involved in neuronal apoptosis (Jacobs et al., 2004). The expression levels of the pro-apoptotic form of p73 are regulated mainly by phosphorylation mediated by the c-Abl kinase. Interestingly, c-Abl is activated by DNA damage, oxidative stress, stress in the endoplasmic reticulum (ER) and in fibers formed by the peptide derived from the amyloid precursor protein (A3 peptide) (Ito et al., 2001; Alvarez et al., 2004). It is therefore possible that c-Abl is activated in diseases where lipid deposits occur such as in NPC disease because NPC disease elicits ER stress and apoptosis (Tessitore et al., 2004; Zhang and Kaufman, 2004; Koh et al., 2006). It is proposed that the activation of TAp73 would induce its translocation from the cytoplasm to the nucleus and an increase in the transcription of its pro-apoptotic target genes such as Puma, Bax, Apaf-1, MDM2, Noxa and Scotin, which in turn activate apoptotic pathways in the ER and mitochondria (Irwin and Miller, 2004; Jacobs at al., 2004; Melino et al., 2004; Ramadan et al., 2005).

The relationship between c-Abl, p73 and Alzheimer's disease (AD) is particularly interesting, considering that NPC disease and AD share common characteristics such as alterations of the neuronal cytoskeleton, the intracellular accumulation of the Aβ peptide, the formation of neurofibrillary tangles in the brain and finally, neurodegeneration (Nixon et al., 2004). Related to this, it has been reported that p73 accumulates in the neuronal nuclei and co-localizes with the neurofibrillary tangles in the brains of AD patients (Wilson et al., 2004). Moreover, polymorphisms in the p73 gene are associated with AD (Li et al., 2004).

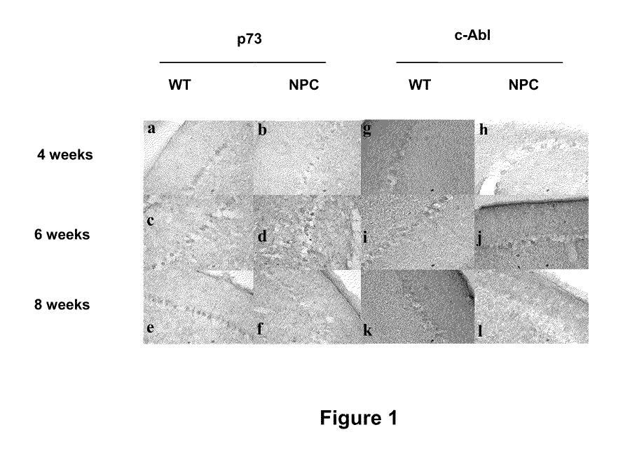

FIG. 1 shows that the c-Abl/p73 system is expressed at high levels in the cerebellum, mainly in Purkinje neurons of wild type mice of all ages, and only at early ages in NPC mice (4 weeks old), because in the mutant mice the expression of both proteins decreases with age due to the loss of Purkinje neurons. In all experiments discussed herein, “NPC mice” refers to mice deficient in the NPC1 protein or NPC1−/−. In FIG. 1, the cerebellums of 4 week old NPC mice and wild type mice are shown at a, b, g and h. The cerebellums of 6 week old NPC mice and wild type mice are shown at c, d, i and j. The cerebellums of 8 week old NPC mice and wild type mice are shown at e, f, k and l. The immunohistochemical procedures utilized were: anti-c-Abl, (K12; 1:500) and anti-p73 (H-79; 1:250) (Santa Cruz Biotech, Santa Cruz, Calif.), and anti-calbindin D-28K (1:1,000). Immunohistochemical procedures were carried out by means of histological stain on floating sections. Immunocytochemistry was performed using the avidin biotin horseradish peroxidase complex (ABC) method (Vector Laboratories, Burlingame, Calif.) and sections were mounted on gelatin pre-coated slides and air-dried, then dehydrated in graded ethanol and finally covered with Permount.

No changes can be seen in the expression of p73 and c-Abl in wild type mice in FIG. 1. However, the NPC mice in FIG. 1 show that there is a progressive decrease in the expression levels of p73 and c-Abl over time that correlates with the loss of Purkinje neurons as the disease progresses. FIG. 1 shows that the c-Abl/p73 system is present in Purkinje cells, and that under situations of cellular stress such as lipidosis, this system could become activated, resulting in the death of Purkinje neurons.

Additional experiments were performed relating to the involvement of p73 and c-Abl in neuronal death. The variability in weight and locomotor coordination were evaluated in wild type and NPC mice because both weight and locomotor ability are indicators of mouse health. Untreated NPC mice experience weight loss that correlates with their inability to look for food and swallow it. The loss of locomotor coordination in NPC mice correlates with the cerebellar deterioration characteristic of NPC disease, because the cerebrum controls locomotor coordination. Both variables start to decline rapidly in NPC mice from the seventh week of life (Pentchev et al., 1995; Voikar et al., 2002). Therefore, if the imatinib mesylate is able to stop neurodegeneration in this disease, it would be expected that an NPC mouse treated with imatinib mesylate would have higher body weight gain and improved locomotor activity compared to NPC mice without treatment.

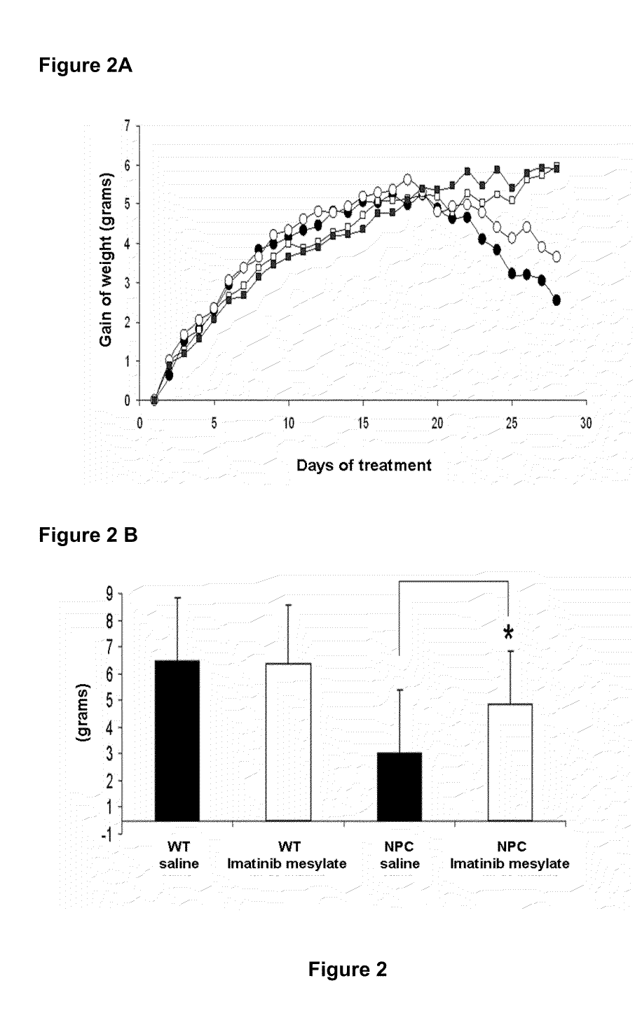

To evaluate the participation of the c-Abl/p73 pathway in vivo, the c-Abl inhibitor Imatinib mesylate was injected intraperitoneally in a daily dose of 50 mg/Kg dissolved in NaCl 0.9% (saline solution) into NPC mice and wild type mice for 28 days beginning when all mice were 4 weeks old. The controls were injected with saline solution and the weight of each mouse was recorded daily, as shown in FIG. 2A. As shown in FIGS. 2A and 2B, imatinib mesylate reduces weight loss in NPC mice. In FIG. 2A, four week old wild type mice (squares) and NPC mice (circles) were treated with saline injection (black squares and circles) or with 50 mg/Kg imatinib mesylate (empty squares and circles) intraperitoneally for 28 days. Mice receiving imatinib mesylate received the drug dissolved in saline solution. The body weight of each mouse was registered daily. FIG. 2A shows body weight variation during treatment. FIG. 2B shows average body weight gain at the end of the treatment with respect to the initial weight with standard deviation on day 28 of treatment. FIGS. 2A and 2B show that NPC mice treated with imatinib mesylate gain more weight (approximately 100%) more than those treated with saline solution.

FIGS. 2A and 2B also show that the weight curves of wild type mice injected with saline or imatinib mesylate are similar, indicating that the drug does not affect the body weight of wild type mice. FIGS. 2A and 2B also make evident that NPC animals tend to increase their weight up to approximately 6 weeks of age (day 15 of treatment), at which point their weight starts to decline. NPC mice, treated with imatinib mesylate lost less weight than NPC mice treated with saline solution; the weight curves for the treated and untreated NPC mice dissociate on day 15 of treatment as seen in FIG. 2A.

This effect becomes clearer when weight gain is plotted on day 28 of treatment as shown in FIG. 2B. This study was done with a large number of animals (wild type mice treated with saline: 19; wild type mice treated with imatinib mesylate: 24; NPC mice treated with saline: 19; NPC mice treated with imatinib mesylate: 18) and demonstrates that imatinib mesylate treatment increases weight gain in NPC mice by approximately 100%. It is important to note that 4 week old NPC mice (the age of all the mice at the beginning of treatment) had the same average body weight as 4 week old wild type mice, as plotted on the graph in FIG. 2A.

The effect of imatinib mesylate treatment on the locomotor activity of mice was also evaluated. To test locomotor activity, the Hanging Test that has been validated for these mice was used (Voikar et al., 2002). For this test a score from 0 to 5 was assigned to the mice based on their motor capabilities. To determine the score, the animal is observed during 30 seconds, a value is assigned according to the following scale: 0, if it falls before 10 seconds; 1, if it hangs from the bar with its fore paws; 2, if it tries to climb on the bar with its hind paws; 3, if it grabs the bar with its fore paws and one or two hind paws; 4, if it grabs the bar with all four paws and its tail; and 5, if it actively escapes from the bar and reaches one of the ends of the bar.

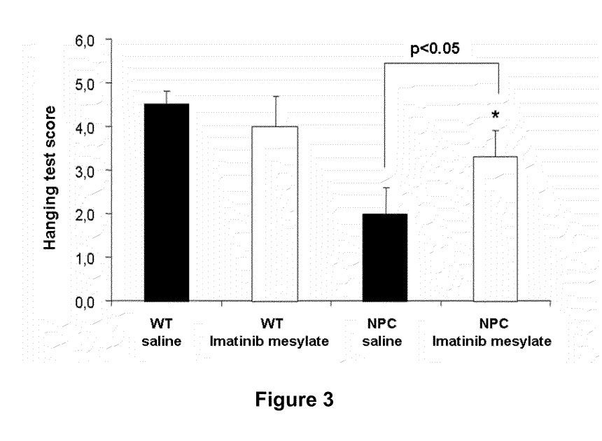

In FIG. 3, the graph shows the quantification of the average scores with standard deviations for the hanging test performed on mice treated with saline injections or imatinib mesylate 50 mg/Kg administered daily intraperitoneally for 28 days. This study was performed with a large number of animals (wild type mice treated with saline: 19; wild type mice treated with imatinib mesylate: 24; NPC mice treated with saline: 19; NPC mice treated with imatinib mesylate: 18).

As shown in FIG. 3, the wild type mice, both those treated with saline solution and those treated with imatinib mesylate, show similar high values in the hanging test, indicating that the drug does not affect the locomotor activity in these mice. NPC mice treated with saline show very low motor coordination, as indicated by the lower scores on the Hanging Test. FIG. 3 also shows that treatment of NPC mice with imatinib mesylate increased the scores in the Hanging Test and decreased the locomotor problems of NPC mice.

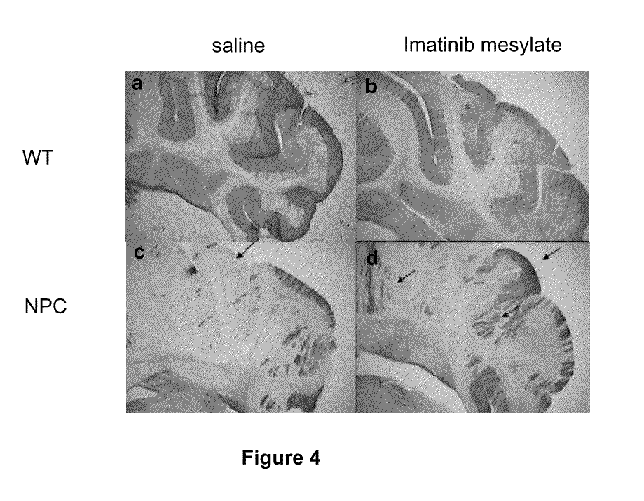

The effect of imatinib mesylate on the survival of the cerebellar Purkinje neurons was also evaluated, because the Purkinje neurons are the most affected cells in NPC disease. FIG. 4 shows the immunohistochemical analysis of calbindin (a specific marker for Purkinje neurons) on cerebellums of wild type (WT) (a, b) and NPC animals (c, d) injected intraperitoneally with saline solution (a, c) and imatinib mesylate 50 mg/Kg (b, d) daily for 28 days. The immunohistochemical protocols used are the same as those described above relating to FIG. 1. It can clearly be seen that the imatinib mesylate treatment reduces the death of Purkinje neurons, when compared with similar cerebellar regions on NPC mice treated with saline solution and imatinib mesylate (indicated with arrows). The density of the neurons is clearly higher in the cerebella of NPC mice treated with imatinib mesylate. Imatinib mesylate treatment therefore reduces the death of Purkinje neurons in NPC mice and does not affect the survival of Purkinje neurons in wild type mice.

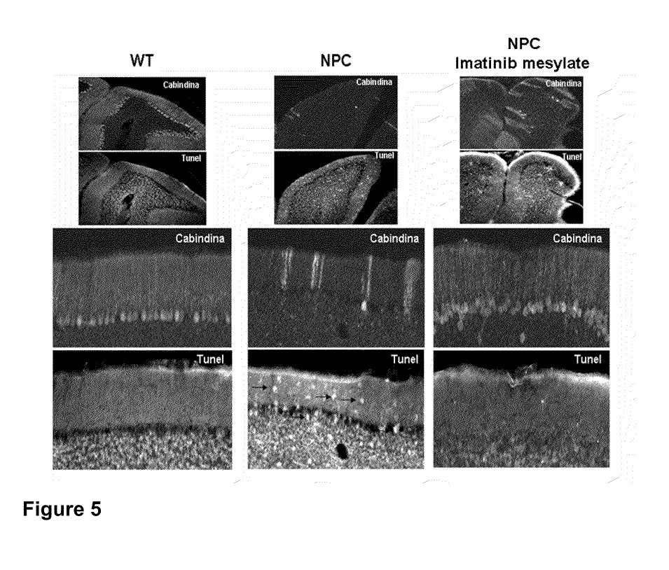

FIG. 5 shows the apoptosis analysis by TUNEL immunofluorescence and calbindin on cerebella from wild type and NPC mice injected intraperitoneally with saline solution or with imatinib mesylate 50 mg/Kg daily for 28 days. The following process was used for in situ apoptosis detection: the terminal deoxynucleotidyl transferase mediated deoxyuridine-biotin (dUTP) nick end labeling (TUNEL) was performed using the apoptosis detection kit purchased from Roche Molecular Biochemicals (Mannheim, Germany). The sections were first incubated in a blocking solution containing 3% H₂O₂ in methanol. Afterward, slices were incubated in 0.1% Triton X-100 in 0.1% sodium citrate over-night at RT to increase the permeability. After being washed twice in PBS, pH 7.4, the sections were immersed in the TUNEL reaction mixture, containing dUTP-fluorescein and terminal deoxynucleotidyl transferase (TdT) conjugated for 60 minutes at 37° C. in a dark, humid atmosphere. The process was terminated by washing the sections twice in PBS buffer. For the immunofluorescence (IF) sections slides were incubated overnight with rabbit polyclonal anti-calbindin D-28K (1:1,000; Chemicon International, Temecula, Calif.). Slices were then incubated with secondary antibodies conjugated with anti-rabbit-Alexa Fluor-594 (Molecular Probes, OR, USA). Fluorescent images were captured with an Olympus BX51 microscope and analyzed with IMAGE pro-express. The immunohistochemical studies for calbindin, a specific Purkinje cell marker, showed a significant increase of the positives areas for Purkinje neurons in NPC mice. Imatinib mesylate treatment does not affect the calbindin staining in wild type mice.

FIG. 5 also illustrates that treatment with imatinib mesylate decreases apoptosis in diverse neuronal types present in the cerebella of NPC mice. The degree of apoptosis was studied by TUNEL immunofluorescence. FIG. 5 shows that the number of bright TUNEL positive cells (apoptotic cells) is lower in the cerebella of NPC mice treated with imatinib mesylate when compared with those treated with saline solution.

It can be clearly seen that the treatment with imatinib mesylate decreases apoptosis in NPC mice, because the number of TUNEL positive cells (bright cells) is significantly less than the number of TUNEL positive cells shown in the NPC mice treated with saline solution.

The results of the experiments described above and shown in FIGS. 2-5, taken together, suggest that the signal transduction pathway c-Abl/p73 participates in apoptosis of Purkinje neurons in NPC disease, and that imatinib mesylate works as a therapeutic agent for the treatment of this disease.

Although the present invention has been described with reference to particular means, materials and embodiments, from the foregoing description one skilled in the art can easily ascertain the essential characteristics of the present invention and various changes and modifications can be made to adapt the various uses and characteristics without departing from the spirit and scope of the present invention as described above.

### REFERENCES

Alvarez A R, Sandoval P C, Leal N R, Castro P U, Kosik K S. Activation of the neuronal c-Abl tyrosine kinase by amyloid-beta-peptide and reactive oxygen species. Neurobiol Dis. 2004; 17:326-36.
Cartsea E D, Morris J A, Coleman K G, Loftus S K, Zhang D, Cummings C, Gu J, et al. Niemann-Pick C1 disease gene: homology to mediators to cholesterol homeostasis. Science. 1997; 277:228-231.
Frolov A, Zielinski S E, Crowley J R, Dudley-Rucker N, Schaffer J E, Ory D S. NPC1 and NPC2 regulate cellular cholesterol homeostasis through generation of low density lipoprotein cholesterol-derived oxysterols. J Biol. Chem. 2003; 278:25517-25.
Huang Z, Hou Q, Cheung N S, Li Q T. Neuronal cell death caused by inhibition of intracellular cholesterol trafficking is caspase dependent and associated with activation of the mitochondrial apoptosis pathway. J. Neurochem. 2006; 97:280-91.
Irwin M S, Miller F D. p73: regulator in cancer and neural development. Cell Death Differ. 2004; 11:S17-22.
Ito Y, Pandey P, Mishra N, Kumar S, Narula N, Kharbanda S, Saxena S, Kufe D. Targeting of the c-Abl tyrosine kinase to mitochondria in endoplasmic reticulum stress-induced apoptosis. Mol Cell Biol. 2001; 21:6233-42.
Jacobs W B, Walsh G S, Miller F D. Neuronal survival and p73/p63/p53: a family affair. Neuroscientist. 2004; 10:443-55.
Koh C H, Qi R Z, Qu D, Melendez A, Manikandan J, Bay B H, Duan W, Cheung N S. U18666A-mediated apoptosis in cultured murine cortical neurons: Role of caspases, calpains and kinases. Cell Signal. 2006 (in press).
Li H, Repa J J, Valasek M A, Beltroy E P, Turley S D, German D C, Dietschy J M. Molecular, anatomical, and biochemical events associated with neurodegeneration in mice with Niemann-Pick type C disease. J Neuropathol Exp Neurol. 2005; 64:323-33.
Li Q, Athan E S, Wei M, Yuan E, Rice S L, Vonsattel J P, Mayeux R P, Tycko B. TP73 allelic expression in human brain and allele frequencies in Alzheimer's disease. BMC Med. Genet. 2004; 5:14.
Liscum L, Klasek J J. Niemann-Pick disease type C. Curr Opin Lipidol. 1998; 9:131-135.
Liscum L, Faust J R. Low density lipoprotein (LDL)-mediated suppression of cholesterol synthesis and LDL uptake is defective in Niemann-Pick type C fibroblasts. J Biol. Chem. 1987; 262:17002-17008.
Melino G, Bernassola F, Ranalli M, Yee K, Zong W X, Corazzari M, Knight R A, Green D R, Thompson C, Vousden K H. p73 Induces apoptosis via PUMA transactivation and Bax mitochondrial translocation. J Biol. Chem. 2004; 279:8076-83.
Naureckiene S, Sleat Delackland H, Fensom A, Vanier M T, Wattiaux R, Jadot M, Lobel P. Identification of HE1 as the second gene of Niemann-PicK C disease. Science. 2000; 290:2298-2301.
Nixon R A. Niemann-Pick Type C disease and Alzheimer's disease: the APP-endosome connection fattens up. Am J. Pathol. 2004; 164:757-61.
Paul C A, Boegle A K, Maue R A. Before the loss: neuronal dysfunction in Niemann-Pick Type C disease. Biochim Biophys Acta. 2004; 1685:63-76.
Pentchev P G, Vanier M T, Suzuki K, Patterson M C. Niemman-Pick disease Type C: a cellular cholesterol lipidosis. In: Scriver C R, Beaudet A L, Sly W S, Valle, D, eds. The metabolic and molecular bases of inherited disease. Volume II. New York. McGraw-Hill, 1995:2625-2640.
Ramadan S, Terrinoni A, Catani M V, Sayan A E, Knight R A, Mueller M, Krammer P H, Melino G, Candi E. p73 induces apoptosis by different mechanisms. Biochem Biophys Res Commun. 2005; 331:713-7.
Reid P C, Sakashita N, Sugii S, Ohno-Iwashita Y, Shimada Y, Hickey W F, Chang T Y. A novel cholesterol stain reveals early neuronal cholesterol accumulation in the Niemann-Pick type C1 mouse brain. J Lipid Res. 2004; 45:582-91.
Steinberg S J, Mondal D, Fensom A H. Co-cultivation of Niemann-Pick disease type C fibroblasts belonging to complementation groups alpha and beta stimulates LDL-derived cholesterol esterification. J Inherit Metab Dis. 1996; 19:769-74.
Sturley S L, Patterson M C, Balch W, Liscum L. The pathophysiology and mechanisms of NP-C disease. Biochim Biophys Acta. 2004; 1685:83-7.
Tessitore A, del P Martin M, Sano R, Ma Y, Mann L, Ingrassia A, Laywell E D, Steindler D A, Hendershot L M, d'Azzo A. GM1-ganglioside-mediated activation of the unfolded protein response causes neuronal death in a neurodegenerative gangliosidosis. Mol. Cell. 2004; 5:753-66.
Voikar V, Rauvala H, Ikonen E. Cognitive deficit and development of motor impairment in a mouse model of Niemann-Pick type C disease. Behav Brain Res. 2002; 132:1-10.
Walkey S U, Suzuki K. Consequences of NPC1 and NPC2 loss of function in mammalian neurons. Biochim Biophys Acta. 2004; 1685:48-62.
Wilson, C., Henry, S., Smith, M. A. & Bowser, R. The p53 homologue p73 accumulates in the nucleus and localizes to neurites and neurofibrillary tangles in Alzheimer disease brain. Neuropathol Appl Neurobiol. 2004; 30:19-29.

Wu Y P, Mizukami H, Matsuda J, Saito Y, Proia R L, Suzuki K. Apoptosis accompanied by up-regulation of TNF-alpha death pathway genes in the brain of Niemann-Pick type C disease. Mol Genet Metab. 2005; 84:9-17.
Zhang K, Kaufman R J. Signaling the unfolded protein response from the endoplasmic reticulum. J Biol. Chem. 2004; 279:25935-8.

1. The use of a therapeutically effective amount of a compound derived from imatinib mesylate or a pharmaceutically acceptable salt thereof to treat neurodegenerative disease.

2. The use of a therapeutically effective amount of a compound derived from imatinib mesylate or a pharmaceutically acceptable salt thereof to attenuate the progress of neurodegeneration in a patient with neurodegenerative disease.

3. The use of a therapeutically effective amount of imatinib mesylate or a pharmaceutically acceptable salt thereof to reduce apoptosis in neurodegenerative diseases.

4. The use of a therapeutically effective amount of imatinib mesylate or a pharmaceutically acceptable salt thereof to treat the neurodegenerative disease Niemann Pick type C(NPC).
